(19) United States
(12) Patent Application Publication
(10) Pub. No.: US 2005/0096594 A1
(43) Pub. Date: May 5, 2005

(54) Cannula arrangements

(75) Inventors: Richard J. Deslauriers, Woodbury, CT (US); Robert T. Potash, Seymour, CT (US)

Correspondence Address: ARENT FOX KINTNER PLOTKIN & KAHN, 1050 CONNECTICUT AVENUE, N.W., SUITE 400, WASHINGTON, DC, 20036, US

(73) Assignee: Doctor's Research Group, Incorporated

(21) Appl. No.: 10/943,001

(22) Filed: Sep. 17, 2004

Related U.S. Application Data
(62) Division of application No. 10/358,347, filed on Feb. 5, 2003.
(60) Provisional application No. 60/376,200, filed on Apr. 30, 2002.

(51) Int. Cl. A61M 5/178

(52) U.S. Cl. 604/164.01

(57) ABSTRACT

A cannula arrangement includes a cannula member and a blunting member. The cannula member includes a hollow elongated member having an opening formed therethrough, and a puncture tip formed at an end of the hollow elongated member. The blunting member is adapted to move relative to the cannula member, and a portion of the blunting member or the entire blunting member is positioned inside the hollow elongated member. Moreover, when a fluid flows between an interior surface of the hollow elongated member and an exterior surface of the blunting member, such as when the puncture tip penetrates a vein of a subject, the fluid is visible via the opening.

This application claims priority from U.S. Provisional Patent Application No. 60/376,200, entitled “Ultra Pressure Curvolinear Geometry With Flashback Indicator,” and filed on Apr. 25, 2002, the disclosure of which is incorporated herein by reference in its entirety.

### BACKGROUND OF THE INVENTION

1. Field of the Invention

The present invention relates generally to cannula arrangements. In particular, the present invention is directed towards cannula arrangements including a cannula member and a blunting member positioned inside the cannula member, in which the cannula member includes a window for viewing blood associated with a successful penetration of a vein of a subject.

2. Description of Related Art

Known cannula, eg., catheter, arrangements, such as the Protective Acuvance® Safety Catheter manufactured by Johnson and Johnson Medical, Inc., which has a principle place of business in Arlington, Tex., may be used to penetrate a vein of a subject. By penetrating the vein of the subject using such known cannula arrangements, a user of the cannula arrangement may draw blood from the subject, intravenously give the subject drugs, intravenously give the subject fluids, or the like.

Referring to FIG. 1, a known cannula arrangement 20, which corresponds to the Protective Acuvance® Safety Catheter, includes a cannula member 1 and a blunting member 13 (shown in FIG. 2c) positioned inside cannula member 1. Cannula member 1 includes an elongated member 2 and a puncturing tip 3 formed at an end of elongated member 2. Cannula arrangement 20 also includes a sheath 14 which surrounds, e.g., encloses, each of elongated member 2 and blunting member 13. Moreover, cannula arrangement 20 includes a hub member 4, a displacement arrangement 8, a flashback chamber 6, and a filter 7. Specifically, hub member 4 is adapted to move in a direction towards puncturing tip 3, e.g., when pressure is applied to a tab 5, and is connected to sheath 14. Displacement arrangement 8 is connected to hub member 4 and is adapted to engage a first end of blunting member 13, and flashback chamber 6 is in fluid communication with elongated member 2. Moreover, filter 7 is adapted to remove air within flashback chamber 6 when blood enters flashback chamber 6.

Referring to FIGS. 2a-2d, in operation, a user 9 of cannula arrangement 20 inserts puncturing tip 3 into a subject or patient 10, e.g., in the vicinity of a vein 11 of subject 10, such that an angle a formed between cannula arrangement 20 and a horizontal plane including vein 11 is within a predetermined angle range. For example, angle a may be between about 35° and about 75°. Referring specifically to FIG. 2b, when puncturing tip 3 successfully penetrates vein 11, a predetermined amount of blood 12 enters flashback chamber 6 via elongated member 2. Subsequently, angle a is reduced to ensure that puncturing tip 3 is positioned within a lumen of vein 11, and to avoid puncturing a vein wall of vein 11. Referring specifically to FIG. 2c, after angle a is reduced, user 9 moves hub member 4 towards puncturing tip 3 by applying pressure to tab 5, which causes sheath 14 to move inside vein 11. Applying pressure to tab 5 also causes displacement arrangement 8 to move towards puncturing tip 3. Displacement arrangement 8 then engages the first end of blunting member 13, which causes a second end of blunting member 13 opposite the first end of blunting member 13 to extend beyond puncturing tip 3. Referring specifically to FIG. 2d, after sheath 14 is positioned inside vein 3, cannula member I and blunting member 13 are removed from sheath 14, such that sheath 14 and hub member 5 remain attached to subject 10. Nevertheless, because the second end of blunting member 13 extends beyond puncturing tip 3, puncturing tip 3 cannot accidentally penetrate user 9 after user 9 uses cannula arrangement 20. Moreover, user 9 then may draw blood from subject 10, intravenously give subject 10 drugs, intravenously give subject 10 fluids, or the like, via hub member 5 and sheath 14.

Nevertheless, as described in an article entitled “Efficacy of Catheter Needles with Safeguard Mechanisms,” authored by Asai et al. (“Asai”), the disclosure of which is incorporated herein by reference in its entirety, in the Protective Acuvance® Safety Catheter, the amount of time which it takes for blood to reach the flashback chamber is delayed. See, e.g., Asai, Page 5, Column 2, Lines 3-6. Consequently, referring to FIG. 3, when puncturing tip 3 penetrates vein 11, by the time the blood reaches the flashback chamber, puncturing tip 3 already may have penetrated through the entire vein 11, which may cause the subject to bruise, and also requires that the user of the cannula arrangement attempt to penetrate a different vein.

### SUMMARY OF THE INVENTION

Therefore, a need has arisen for cannula arrangements which overcome these and other shortcomings of the related art. A technical advantage of the present invention is that in a cannula arrangement having a cannula member and a blunting member positioned inside the cannula member, an opening may be formed through an elongated member of the cannula member, and the opening provides a visual indication that a puncturing tip of the cannula member successfully has penetrated a vein of a subject. Consequently, visual indication that the puncturing tip successfully has penetrated the vein may be given to a user of the cannula arrangement before the puncturing tip penetrates the entire vein.

According to an embodiment of the present invention, a cannula arrangement is described. The cannula arrangement comprises a cannula member, and the cannula member comprises a hollow elongated member having at least one opening formed therethrough. The cannula member also comprises a puncture tip formed at an end of the hollow elongated member. The cannula arrangement also comprises a blunting member adapted to move relative to the cannula member, and at least a portion of the blunting member is positioned inside the hollow elongated member. Moreover, when a fluid, e.g., blood, flows between an interior surface of the hollow elongated member and an exterior surface of the blunting member, the fluid is visible via the at least one opening.

According to another embodiment of the present invention, a cannula arrangement is described. The cannula arrangement comprises a cannula member, and the cannula member comprises a hollow elongated member having at least one first opening formed therethrough. The cannula member also comprises a puncture tip formed at an end of the hollow elongated member. The cannula arrangement also comprises a hollow blunting member having at least one second opening formed therethrough. The hollow blunting member is adapted to move relative to the cannula member, and at least a portion of the blunting member is positioned inside the hollow elongated member. Moreover, when a fluid, e.g., blood, flows inside the hollow blunting member, the fluid is visible via the at least one first opening and the at least one second opening.

According to yet another embodiment of the present invention, a cannula arrangement is described. The cannula arrangement comprises a cannula member, and the cannula member comprises a hollow elongated member having at least one opening formed therethrough. The cannula member also comprises a puncture tip formed at an end of the hollow elongated member, and the puncture tip is adapted to penetrate a vein of a subject. Moreover, when the puncture tip penetrates the vein of the subject, blood flows between an interior surface of the hollow elongated member and an exterior surface of the blunting member, such that the blood is visible to a user of the cannula arrangement via the at least one opening. The cannula arrangement also comprises a blunting member adapted to move relative to the cannula member, and at least a portion of the blunting member is positioned inside the hollow elongated member. The cannula arrangement further comprises a translucent member surrounding at least a portion of the hollow elongated member, and a hub member connected to the translucent member. The hub member is adapted to move in a direction towards the puncture tip. Moreover, the cannula arrangement also comprises a displacement arrangement operationally connected to the hub member. Specifically, when the hub member moves in the direction towards the puncture tip, the translucent member enters the vein of the subject and a portion of the blunting member extends beyond the puncture tip, such that when the cannula member and the blunting member are removed from the translucent member, the puncture tip does not penetrate the user of the cannula arrangement.

Other objects, features, and advantages of the present invention will be apparent to persons of ordinary skill in the art in view of the foregoing detailed description of the invention and the accompanying drawings.

### BRIEF DESCRIPTION OF THE DRAWINGS

For a more complete understanding of the present invention, needs satisfied thereby, and the objects, features, and advantages thereof, reference now is made to the following description taken in connection with the accompanying drawings.

FIG. 1 is a cross-sectional view of a known cannula arrangement.

FIGS. 2a-2d are cross-sectional views depicting an operation of the known cannula arrangement.

FIG. 3 is cross-section view depicting the known cannula arrangement of FIG. 1 penetrating an entire vein of a subject.

FIG. 4 is a cross-sectional view of a cannula arrangement according to an embodiment of the present invention.

FIG. 5 is a cross-sectional view of a cannula member and blunting member according to an embodiment of the present invention.

FIG. 6 is a cross-sectional view of a cannula member and blunting member according to another embodiment of the present invention.

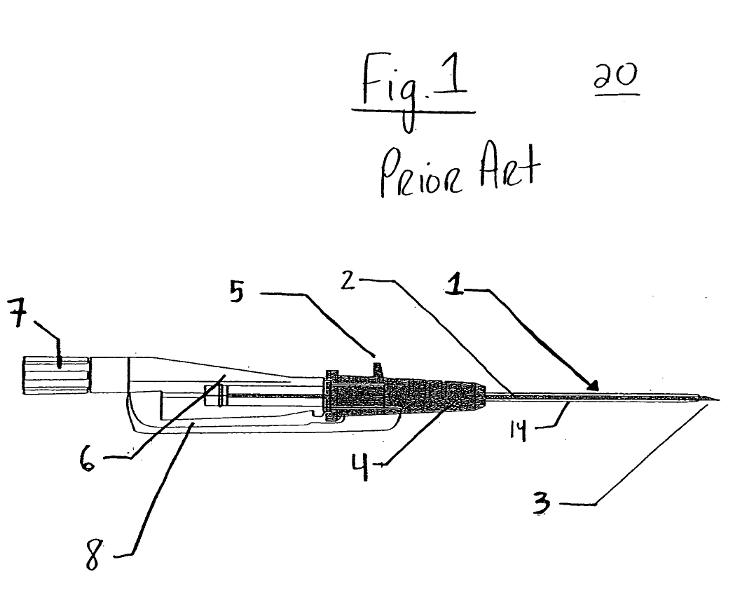

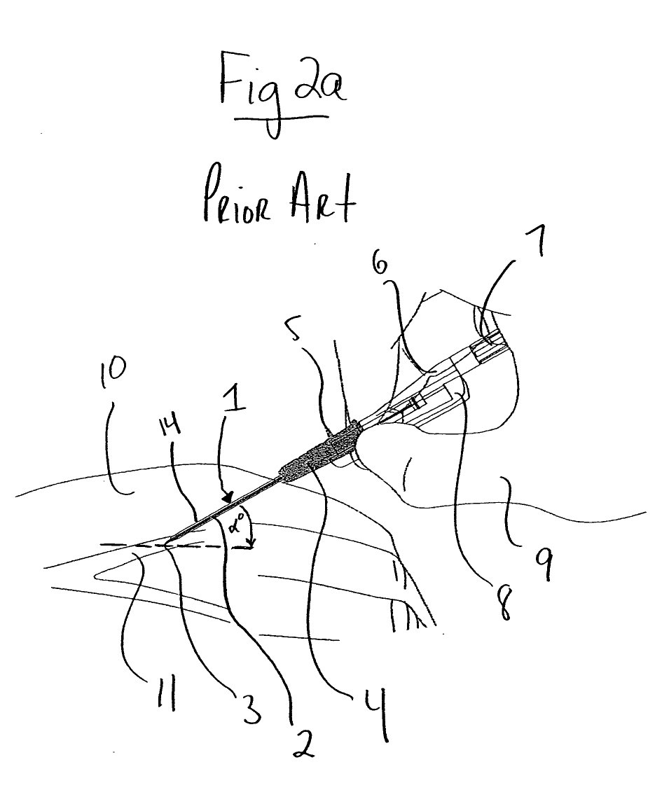

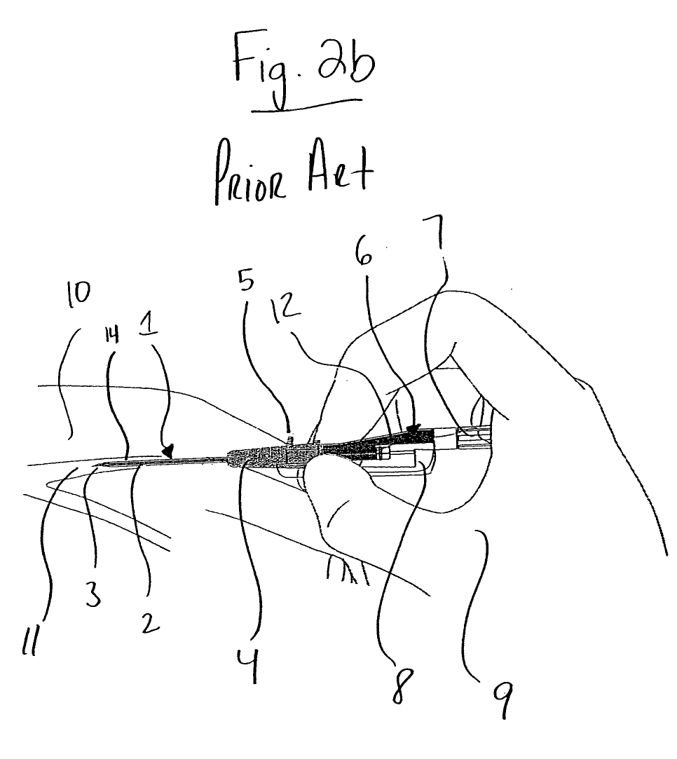

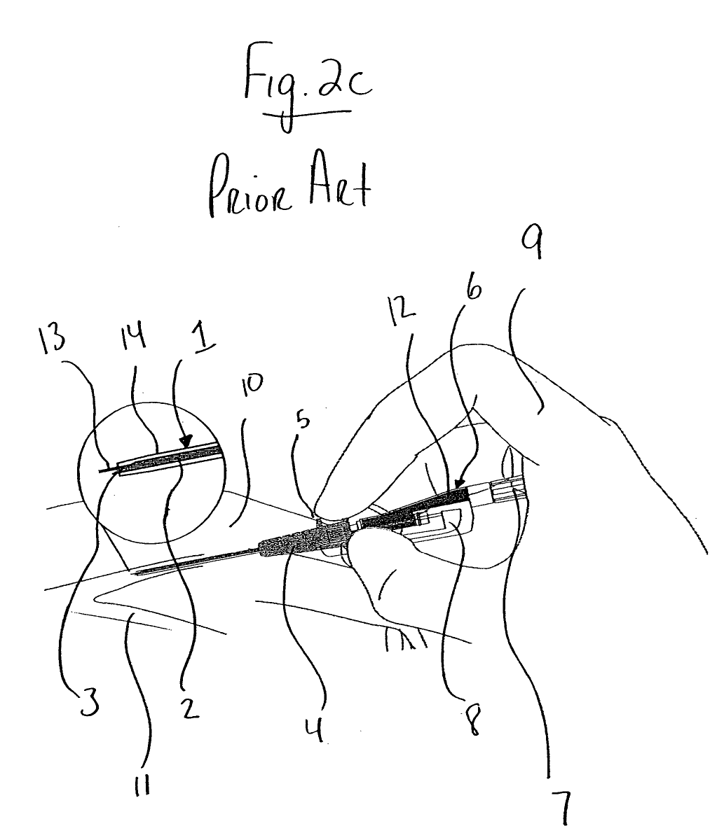

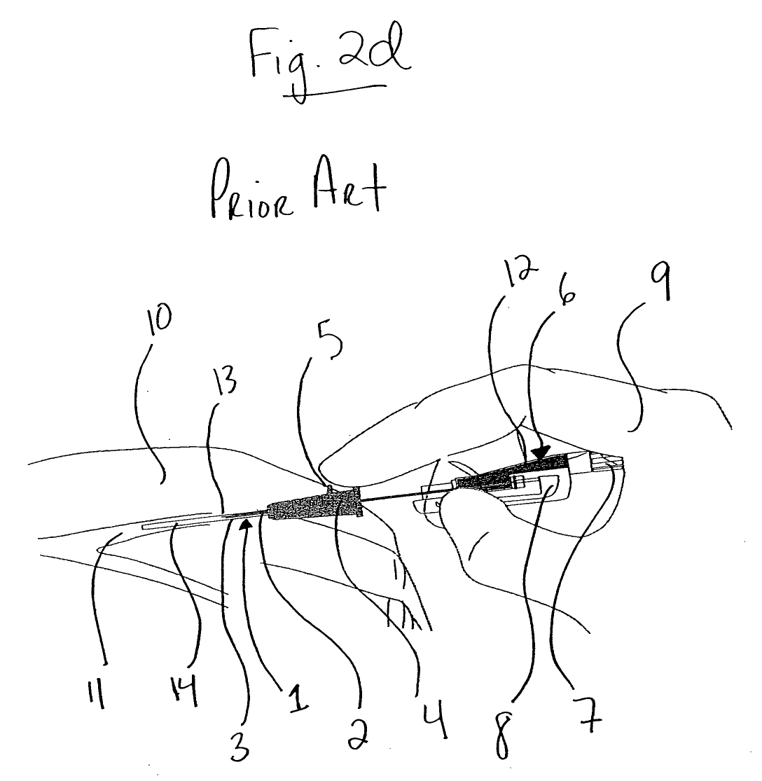

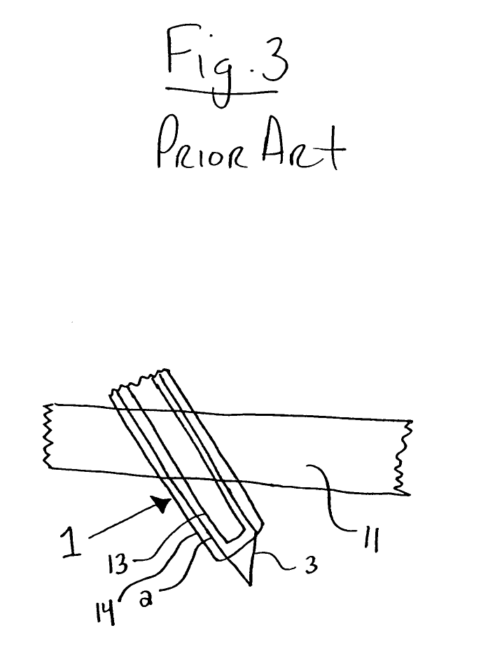

### DETAILED DESCRIPTION OF THE DRAWINGS

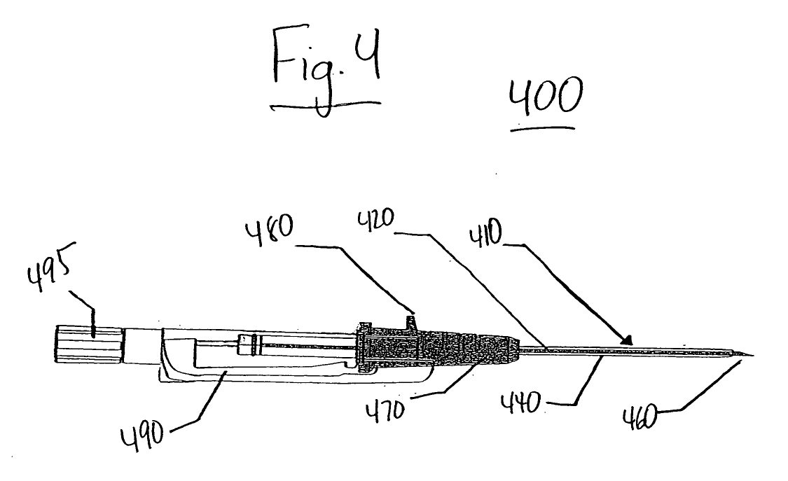

Preferred embodiments of the present invention and their advantages may be understood by referring to FIGS. 4-6, like numerals being used for corresponding parts in the various drawings.

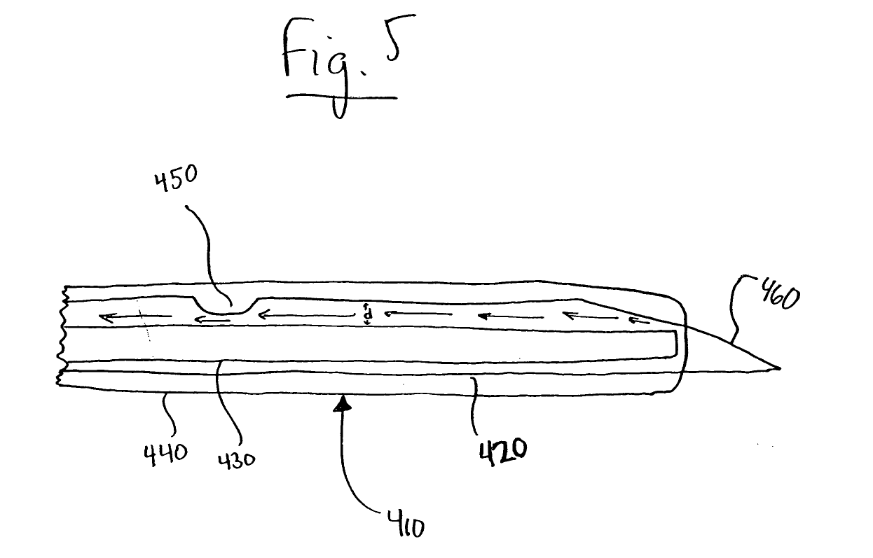

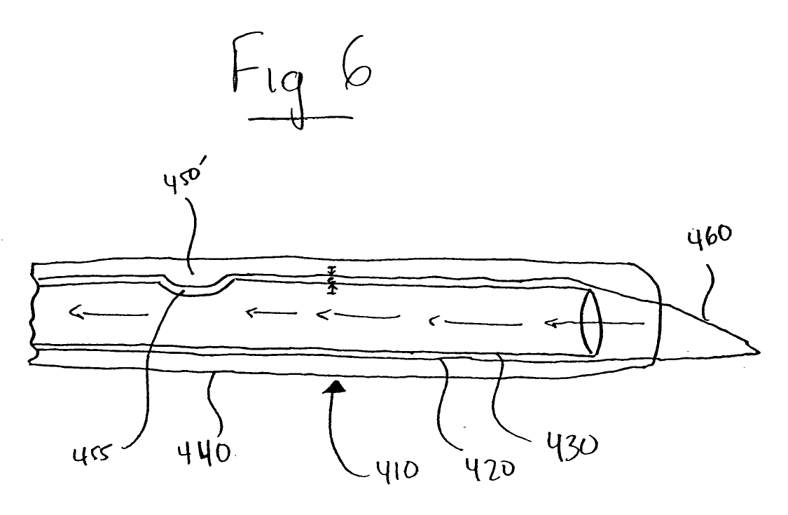

Referring to FIG. 4, a cannula arrangement 400 according to an embodiment of the present invention is depicted. Cannula arrangement 400 may comprise a cannula member 410 and a blunting member 430 (shown in FIGS. 5 and 6), and at least a portion of blunting member 430 may be positioned inside cannula member 410. Specifically, cannula member 410 may comprise an elongated member 420 and a puncturing tip 460 formed at an end of elongated member 420. Elongated member 420 may be hollow, and at least a portion of blunting member 430 may be positioned inside elongated member 420. In an exemplary embodiment of the present invention, elongated member 420 and/or blunting member 430 may be manufactured from AISI type 304 stainless steel. Moreover, it will be understood by those of ordinary skill in the art that puncturing tip 460 may have various cross-sectional shapes, eg., a concave cross-sectional shape, a convex cross-sectional shape, a two-prong cross-sectional shape, a serrated cross-sectional shape, or the like. Cannula arrangement 400 also may comprise a translucent member 440, eg., a translucent sheath, which surrounds, e., encloses, at least a portion of each of elongated member 420 and blunting member 430. Moreover, cannula arrangement 400 may comprise a hub member 470, a displacement arrangement 490, and a filter 495. Specifically, hub member 470 may be adapted to move in a direction towards puncturing tip 460, e.g., when pressure is applied to a tab 480, and may be connected to translucent member 440. Displacement arrangement 490 may be operationally connected to hub member 470, and may be adapted to engage a first end of blunting member 430. For example, displacement arrangement 490 may engage hub member 470. Moreover, filter 495 may be adapted to remove air within cannula arrangement 400 when a fluid, e.g., blood, enters cannula arrangement 400.

Referring to FIG. 5, in an embodiment of the present invention, elongated member 420 may have at least one opening 450 formed therethrough. For example, at least a portion of opening 450 may have a curved cross-sectional shape, and opening 450 may be formed using wire electrical discharge machining (“EDM”) technology. Moreover, a sealing member (not shown) may be positioned within opening 450. For example, the sealing member may be translucent, may be adapted to change from a first color to a second color when the fluid contacts the sealing member, or the like. In this embodiment, blunting member 430 may be a substantially solid blunting member, and an interior surface of elongated member 420 and an exterior surface of blunting member 430 may be separated by a first separation distance (d). As shown by the arrows in FIG. 5, first separation distance (d) may be selected, such that the fluid, e.g., blood, may flow between the interior surface of elongated member 420 and the exterior surface of blunting member 430. When the fluid reaches opening 450, the fluid is visible via opening 450 and translucent member 440. As such, in one exemplary embodiment of the present invention, a flashback chamber is not used, which reduces the amount of material used to manufacture cannula arrangement 400. Moreover, the amount of air to be removed from cannula arrangement 400 may be reduced relative to known cannula arrangements which include a flashback chamber.

Referring to FIG. 6, in another embodiment of the present invention, elongated member 420 may have at least one first opening 450′ formed therethrough, and blunting member 430 may have at least one second opening 455 formed therethrough, e.g., substantially vertically aligned with first opening 450′. For example, at least a portion of first opening 450′ and/or at least a portion of second opening 455 may have a curved cross-sectional shape, and first opening 450′ and/or second opening 455 may be formed using wire EDM technology. Moreover, a sealing member (not shown) may be positioned within first opening 450′ and/or second opening 455. For example, the sealing member may be translucent, may be adapted to change from a first color to a second color when the fluid contacts the sealing member, or the like. In this embodiment, blunting member 430 may be a hollow blunting member, and an interior surface of elongated member 420 and an exterior surface of blunting member 430 may be separated by a second separation distance (e). As shown by the arrows in FIG. 6, second separation distance (e) may be less than first separation distance (d). Specifically, second separation distance (e) may be minimized, such that the fluid, e.g., blood, may flow between inside blunting member 430. Moreover, the interior surface of elongated member 420 and the exterior surface of blunting member 430 may be lubricated, thereby creating a seal therebetween, which may prevent the fluid from flowing between the interior surface of elongated member 420 and the exterior surface of blunting member 430. When the fluid reaches first opening 450′ and second opening 455, the fluid is visible via second opening 455, first opening 450′, and translucent member 440.

In any of the above-described embodiments of the present invention, in operation, a user of cannula arrangement 400 may insert puncturing tip 460 into a subject or patient, e.g., in the vicinity of a vein of the subject, such that an angle formed between cannula arrangement 400 and a horizontal plane including the vein is within a predetermined angle range. For example, the angle may be between about 35° and about 75°. When puncturing tip 460 successfully penetrates the vein, the fluid, e.g., blood, may flow between the exterior surface of blunting member 430 and the interior surface of elongated member 420, such that the fluid may be viewed by the user of cannula arrangement 400 via opening 450 and translucent member 440. Alternatively, when blunting member 430 is a hollow blunting member, the fluid may flow within blunting member 430, such that the fluid may be viewed by the user of cannula arrangement 400 via first opening 450′, second opening 455, and translucent member 440. Subsequently, the angle formed between cannula arrangement 400 and the horizontal plane including the vein may be reduced to increase a likelihood that puncturing tip 460 is positioned within a lumen of the vein, and to reduce a likelihood of puncturing a vein wall of the vein. After the angle formed between cannula arrangement 400 and the horizontal plane including the vein is reduced, the user of cannula arrangement 400 may move hub member 470 towards puncturing tip 460, e.g., by applying pressure to tab 480, which causes translucent member 440 to move inside the vein. Applying pressure to tab 480 also causes displacement arrangement 490 to move towards puncturing tip 460. Displacement arrangement 490 then may engage the first end of blunting member 430, which causes a second end of blunting member 430 opposite the first end of blunting member 430 to extend beyond puncturing tip 460. After translucent member 440 is positioned inside the vein, cannula member 410 and blunting member 430 may be removed from translucent member 440, such that translucent member 440 and hub member 470 may remain attached to the subject. Nevertheless, because the second end of blunting member 430 extends beyond puncturing tip 460, puncturing tip 460 cannot accidentally penetrate the user of cannula arrangement 400 after using cannula arrangement 400. The user of cannula arrangement 400 then may draw blood from the subject, intravenously give the subject drugs, intravenously give the subject fluids, or the like, via hub member 470 and translucent member 440. Moreover, because opening 450 is formed through elongated member 420, the fluid is more rapidly visible to the user of cannula arrangement 400 than in the known cannula arrangements.

Specifically, in a cannula arrangement which does not include a blunting member, blood readily may flow to the flashback chamber because there are no obstructions inside the elongated member of the cannula member. Forming an opening for viewing the fluid through the elongated member weakens the elongated member relative to when no openings for viewing the fluid are formed through the elongated member. Moreover, it may be easier and more economical to manufacture a flashback chamber than it is to form an opening for viewing the fluid through the elongated member. Further, the time differential between the fluid reaching the flashback chamber and the fluid reaching the opening is minimal when there are no obstructions inside the elongated member. As such, when no obstructions are present in the elongated member, it is not desirable to have an opening formed through the elongated member because the fluid readily may reach the flashback chamber, and the flashback chamber is easier to manufacture and costs less to manufacture. Nevertheless, the blunting member covers at least 50% of the area inside the elongated member. Consequently, the amount of time which it takes for the fluid to reach the flashback chamber is significantly greater relative to when the blunting member is not present. By forming the opening for viewing the fluid through the elongated member, the distance which the fluid travels before it is viewed by the user of the cannula arrangement is significantly reduced relative to when a flashback chamber used. As such, the amount of time which it takes for the fluid to reach the opening is significantly less than the amount of time which it takes for the fluid to reach the flashback chamber. Moreover, this significant time difference makes it desirable to form the opening for viewing the fluid in the elongated member despite the above-described weakening of the elongated member by such opening.

It will be readily understood by those of ordinary skill in the art that opening 450 may used in any known cannula arrangement which uses a blunting member, by replacing the elongated member of such known cannula arrangements with elongated member 420. Similarly, it will be readily understood by those of ordinary skill in the art that first opening 450′ and second opening 455 may used in any known cannula arrangement which uses a blunting member, by replacing the elongated member and blunting member of such known cannula arrangements with elongated member 420 and blunting member 430, respectively.

While the invention has been described in connecting with preferred embodiments, it will be understood by those of ordinary skill in the art that other variations and modifications of the preferred embodiments described above may be made without departing from the scope of the invention. Other embodiments will be apparent to those of ordinary skill in the art from a consideration of the specification or practice of the invention disclosed herein. It is intended that the specification and the described examples are considered as exemplary only, with the true scope and spirit of the invention indicated by the following claims.

1-11. (canceled)

12. A cannula arrangement, comprising:
a cannula member, wherein the cannula member comprises:
a hollow elongated member having first opening formed therethrough; and
a puncture tip formed at an end of the hollow elongated member; and
a hollow blunting member adapted to move relative to the cannula member, wherein at least a portion of the blunting member is hollow between a first end of the blunting member and a second end of the blunting member, wherein at least a portion of the blunting member is positioned inside the hollow elongated member, and wherein when a fluid flows between an exterior surface of the blunting member and an interior surface of the hollow elongated member, the fluid is visible via the first opening.

13. The cannula arrangement of claim 12, further comprising a translucent member surrounding at least a portion of the hollow elongated member.

14. The cannula arrangement of claim 13, further comprising:
a hub member connected to the translucent member, wherein the hub member is adapted to move in a direction towards the puncture tip; and
a displacement arrangement operationally connected to the hub member.

15. The cannula arrangement of claim 14, wherein when the hub member moves in the direction towards the puncture tip, the hub member moves the translucent member in the direction towards the puncture tip.

16. The cannula arrangement of claim 15, wherein when the hub member moves in the direction towards the puncture tip, the displacement arrangement engages a first end of the hollow blunting member, and moves the hollow blunting member relative to the hollow elongated member.

17. The cannula arrangement of claim 16, wherein the displacement arrangement moves the hollow blunting member in the direction toward the puncture tip, such that a second end of the hollow blunting member extends beyond the puncture tip.

18. The cannula arrangement of claim 17, further comprising a filter adapted to remove air from inside the cannula arrangement when the fluid enters the hollow elongated member.

19. The cannula arrangement of claim 12, wherein at least one of the at least one first opening and the at least one second opening has a curved cross-sectional area.

20. The cannula arrangement of claim 12, further comprising a sealing member positioned within the at least one second opening.

21. The cannula arrangement of claim 20, wherein the sealing member is translucent.

22. The cannula arrangement of claim 20, wherein the sealing member is adapted to change from a first color to a second color when the fluid contacts the sealing member.

23. (canceled)

24. The cannula arrangement of claim 12, wherein the entire blunting member is hollow between the first end of the blunting member and the second end of the blunting member.

25. A cannula arrangement, comprising:
a cannula member, wherein the cannula member comprises:
a hollow elongated member having a first opening formed therethrough; and
a puncture tip formed at an end of the hollow elongated member; and
a blunting member having a second opening formed therethrough, wherein the blunting member is adapted to move relative to the cannula member, wherein at least a portion of the blunting member is hollow between a first end of the blunting member and a second end of the blunting member, wherein at least a portion of the blunting member is positioned inside the hollow elongated member, and wherein when a fluid flows inside the blunting member, the fluid is visible via the first opening and the second opening.

26. The cannula arrangement of claim 25, wherein the entire blunting member is hollow between the first end of the blunting member and the second end of the blunting member.
